(19) United States
(12) Patent Application Publication
(10) Pub. No.: US 2024/0430601 A1
(43) Pub. Date: Dec. 26, 2024

(54) Acoustic Ear-Hook

(71) Applicant: Yuanhao Liu, Shenzhen (CN)

(72) Inventor: Yuanhao Liu, Shenzhen (CN)

(21) Appl. No.: 18/241,900

(22) Filed: Sep. 4, 2023

(30) Foreign Application Priority Data
Jun. 20, 2023 (CN) 2023107351169

(51) Int. Cl. H04R 1/10; A61B 5/00; A61B 5/024

(52) U.S. Cl. CPC H04R 1/1016; A61B 5/02438; A61B 5/02444; A61B 5/6816; H04R 1/1041; H04R 1/105; H04R 1/1066; H04R 2420/07

(57) ABSTRACT

An acoustic ear-hook has the advantage that it can simultaneously have the decorative effect of an earring and can also provide the function of an earphone. The acoustic ear-hook includes an earphone assembly and a hooking portion connected to the earphone assembly for hooking the acoustic ear-hook to a user's ear. Preferably, the earphone assembly comprises a decorative shell, an acoustic part and a control part, wherein the acoustic part is electrically connected to the control part for generating sound, and the acoustic part and the control part are respectively installed inside the decorative shell, the hooking portion is connected to the decorative shell.

### CROSS REFERENCE OF RELATED APPLICATION

This application is a non-provisional application that claims priority under 35 U.S.C. § 119 to China application number CN202310735116.9, filing date Jun. 20, 2023, wherein the entire content of which is expressly incorporated herein by reference.

### BACKGROUND OF THE PRESENT INVENTION

### Field Of Invention

The present invention relates to an acoustic earphone, and more particularly to an acoustic ear-hook with a decorative effect.

### Description Of Related Arts

Earrings, which are common accessories in people's daily lives, are mainly used to decorate and beautify the ear parts of a human body and enhance self-image expression. However, the conventional earrings have only decorative functions and cannot meet the diversified needs of modern people.

Earphones, which are common audio playing devices, are generally used to play audio when being connected to electronic devices (e.g. mobile phones, computers, etc.). The advantage of earphones is that they can provide a personalized music experience to a user and avoid an influence on the surrounding environment. However, the way earphones are worn often conflicts with the way earrings are worn, so that they cannot be worn at the same time, making it difficult for people to show their decorations while enjoying music or answering calls.

In addition, with the development of technology, wireless technology is becoming more and more mature, and wireless earphones have been widely used in various scenarios. However, these devices are usually designed to be relatively simple, and lacked a sense of fashion, which cannot meet people's needs for beauty. At the same time, because the way of wearing earphones is usually in conflict with the way of wearing earrings, people cannot wear earrings while using earphones, or affect the display of earrings, which undoubtedly limits people's wear choices.

At present, there are some technologies that try to combine the functions of earrings and earphones, such as Bluetooth headphones designed as accessories that can be worn on the ear, but these products often have defects in aesthetics or functionality. For example, because of the need to embed batteries, audio receivers and other components, the volume of the earrings are resulted to be bulk in size, or because of the attention to the decorative earrings, the sound quality is greatly reduced.

Moreover, the current decorative earphones will lose their decorative function when the user takes off the earphones and does not use them. However, if the user continues to wear them to maintain the decorative effect, it will affect the user's ability to hear external sounds and pose a certain safety hazard.

### SUMMARY OF THE PRESENT INVENTION

The invention is advantageous in that it provides an acoustic ear-hook which can simultaneously have the decorative effect of an earring and can also provide the function of an earphone.

Another advantage of the present invention is to provide an acoustic ear-hook, wherein an acoustic part of the acoustic ear-hook can be disposed in a cavum conchae of a user to provide a semi-in-ear audio function.

Another advantage of the present invention is to provide an acoustic ear-hook which is adapted to be inverted and hooked on an earlobe when the audio function is not in use, so as to provide the function of the decoration.

Another advantage of the present invention is to provide an acoustic ear-hook which can be continuously suspended on the ear to provide decorative functions when the audiol function is not used.

Another advantage of the present invention is to provide an acoustic ear-hook which can provide a non-in-ear audio function when it is inverted and hooked on the user's earlobe.

Another advantage of the present invention is to provide an acoustic ear-hook which can be worn in a non-in-ear manner and provide the non-in-ear audio function while providing the decorative function.

Another advantage of the present invention is to provide an acoustic ear-hook which can be freely switched between a non-in-ear wearing mode and a semi-in-ear wearing mode to provide different audio functions without the need to remove the acoustic ear-hook.

Another advantage of the present invention is to provide an acoustic ear-hook which is suitable for wearing for a long time without discomfort in the non-in-ear mode.

Another advantage of the present invention is to provide an acoustic ear-hook, wherein the acoustic ear-hook can be worn in a semi-in-ear mode, which has better sound effect, does not affect others, and privacy will not be eavesdropped.

Another advantage of the present invention is to provide an acoustic ear-hook, wherein the acoustic part of the acoustic ear-hook placed into the cavum conchae is separately designed, and the acoustic part can only accommodate the speaker, which can make the speaker larger in size and better in sound quality.

Another advantage of the present invention is to provide an acoustic ear-hook, wherein the acoustic ear-hook increases the size of the mid-low moving coil loudspeaker, or the moving iron loudspeaker, and the loudspeaker is close to the ear canal, which can enhance the audio effect.

Another advantage of the present invention is to provide an acoustic ear-hook, wherein the acoustic ear-hook comprises a microphone module, which can be used to receive the user's voice, and can realize the function of a telephone.

Another advantage of the present invention is to provide an acoustic ear-hook, wherein the acoustic ear-hook can provide the function of a hearing-aid to help the user with poor hearing.

Another advantage of the present invention is to provide an acoustic ear-hook, wherein the acoustic ear-hook can be freely switched between a phone mode and a hearing-aid mode, when a phone call comes in, the earphone can be connected and the acoustic ear-hook is switched to the phone mode; at ordinary times, the acoustic ear-hook, which can be used as the hearing-aid, is convenient for the users with poor hearing to wear and can avoid the trouble caused by the replacement of earphones and hearing-aid.

Another advantage of the present invention is to provide an acoustic ear-hook, wherein the acoustic ear-hook comprises an artificial intelligence module which allows the acoustic ear-hook to provide the function of an artificial intelligence assistant.

Another advantage of the present invention is to provide an acoustic ear-hook, wherein the acoustic ear-hook comprises a heart rate detection module, such as a photoplethysmography (PPG), to measure the optical changes in the blood in response to the pulse, so as to estimate the heart rate.

Another advantage of the present invention is to provide an acoustic ear-hook, wherein the acoustic ear-hook can adjust and improve the user's mood, and adjust the user's state to make the user feel happy by monitoring the user's heart rate and other mood values, and then playing preset music or imitating prese sound.

Another advantage of the present invention is to provide an acoustic ear-hook, wherein the guardian can obtain the heart-rate related data through external software to obtain the feedback of the user's state and respond to the user's adverse conditions in a timely manner.

Another advantage of the present invention is to provide an acoustic ear-hook, wherein has a plurality of decorative surfaces which can be designed or added with decorative elements, providing the user with a variety of decorative options.

Another advantage of the present invention is to provide an acoustic ear-hook which can use processes such as diamond inlaying, patterning, and carving on the decorative surface to make the headphones more decorative.

According to the present invention, the foregoing and other objects and advantages are attained by an acoustic ear-hook comprising an earphone assembly and a hooking portion, wherein the earphone assembly is connected to the hooking portion for providing an acoustic effect, the hooking portion is provided for hooking the acoustic ear-hook to a user's ear.

Preferably, the earphone assembly comprises a decorative shell, an acoustic part and a control part, wherein the acoustic part is electrically connected to the control part to provide the acoustic effect under control of the control part, wherein the acoustic part and the control part are respectively installed inside the decorative shell, and the hooking portion is connected to the decorative shell.

Preferably, the decorative shell comprises a first decorative body, a second decorative body and a connecting element, wherein the first decorative body and the second decorative body are respectively connected to two ends of the connecting element, and the acoustic part is positioned inside the first decorative body in a manner which is capable of outwardly emitting sound, wherein the control part is positioned inside the second decorative body.

Preferably, the first decorative body comprises a first shell and a first lid, wherein the first decorative body also has a first accommodating chamber and comprises a first decorative surface, wherein the first lid is assembled with the first shell in a manner which is capable of transmitting the sound, wherein the first accommodating chamber is formed inside the first lid and the first shell, and the first decorative surface is formed on an outside surface of the first decorative body, the acoustic part is positioned in the first accommodating chamber, and an acoustic side of the acoustic part is positioned towards the first lid.

Preferably, the second decorative body comprises a second shell and a second lid, wherein the second decorative body has a second accommodating chamber and comprises a second decorative surface, wherein the second lid is assembled to the second shell, wherein the second accommodating chamber is formed inside the second shell and the second lid, and the second decorative surface is formed on an outside surface of the second decorative body, wherein the control part is positioned in the second accommodating chamber.

Preferably, the connecting element comprises a connecting body, a first end portion and a second end portion, wherein the connecting element further has a wire groove and comprises a third decorative surface, wherein the first end portion and the second end portion are respectively formed at two ends of the connecting body, wherein the wire groove is indented in the connecting body, and the third decorative surface is formed on an outside surface of the connecting element, wherein the first end portion is connected to the first shell of the first decorative body, and the second end portion is connected to the second shell of the second decorative body.

Preferably, the hooking portion comprises a hooking body which is connected to the second decorative body, and a hooking space is formed between the hooking body and the second decorative body.

Preferably, the hooking body comprises an entry end, an adjustment part and a connecting end, wherein the connecting end is connected to the second decorative body, one end of the adjustment part is connected to the connecting end, the other end of the adjustment part is connected to the entry end.

Preferably, the first decorative body is adapted to be placed at a cavum conchae of the ear and abutted against an antitragus.

Preferably, at least part of the decorative shell is provided with decorative elements by diamond inlaying, patterning, engraving or detachable assembly of an ornament.

Preferably, the control part comprises a control circuit, a power module, a communication module and a charging module, wherein the control circuit is electrically connected to the power module, the communication module is electrically connected to the control circuit, and the charging module is rechargeable electrically connected to the control circuit, wherein the acoustic part is electrically connected to the control circuit to provide the acoustic effect under control of the control circuit.

Preferably, the control part further comprises a microphone unit which is electrically connected to the control circuit in a manner which is capable for receiving voice of the user.

Preferably, the control part further comprises an artificial intelligence module electrically connected to the control circuit.

Preferably, the control part further comprises a heart rate detection module electrically connected to the control circuit.

Preferably, the acoustic ear-hook integrates functions of hearing-aid and earphone.

Preferably, the earphone assembly comprises a decorative shell, an acoustic part and a control part, wherein the acoustic part is electrically connected to the control part, wherein the decorative shell comprises a first decorative body, a second decorative body and a connecting element which is extended between the first decorative body and the second decorative body, wherein the acoustic part is disposed ins the first decorative body which is capable of being placed at a cavum conchae of the ear for enabling the acoustic part to provide the acoustic effect to the user's ear and being hung under an earlobe of the ear when the hooking portion is hooked on the ear.

Preferably, when the first decorative body is placed at the cavum conchae, the hooking portion and the second decorative body define a hooking space for clamping the earlobe of the user.

Preferably, when the hooking portion is penetrating the earlobe of the user for retaining the second decorative body on the earlobe, the first decorative body is hung under the first decorative body.

Preferably, the control part is disposed in the second decorative body.

Preferably, at least one of the first decorative body, the connecting element and the second decorative body is provided with decorative elements by diamond inlaying, patterning, engraving or detachable assembly of an ornament.

### BRIEF DESCRIPTION OF THE DRAWINGS

FIG. 1 and FIG. 2 are perspective views of an acoustic ear-hook according to a first preferred embodiment of the present invention.

FIG. 3 is a side view of the acoustic ear-hook according to the first preferred embodiment of the present invention.

FIG. 4 and FIG. 5 are exploded views of the acoustic ear-hook according to the first preferred embodiment of the present invention.

FIG. 6 and FIG. 7 are schematic views illustrating a using method of the acoustic ear-hook according to the first preferred embodiment of the present invention.

FIG. 8 is a schematic view of decorative surfaces of the acoustic ear-hook according to the first preferred embodiment of the present invention.

FIG. 9 and FIG. 10 are perspective views illustrating the using method of an acoustic ear-hook according to a second preferred embodiment of the present invention.

FIG. 11 is a side view of the acoustic ear-hook according to the second preferred embodiment of the present invention.

FIG. 12 and FIG. 13 are exploded views of the acoustic ear-hook according to the second preferred embodiment of the present invention.

FIG. 14 is a schematic view illustrating a using method of the acoustic ear-hook according to the second preferred embodiment of the present invention.

FIG. 15 is a schematic view of a decorative surface of the acoustic ear-hook according to the second preferred embodiment of the present invention.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

Those skilled in the art should understand that, in the disclosure of the present invention, terminologies of “longitudinal,” “lateral,” “upper,” “front,” “back,” “left,” “right,” “perpendicular,” “horizontal,” “top,” “bottom,” “inner,” “outer,” and etc. that indicate relations of directions or positions are based on the relations of directions or positions shown in the appended drawings, which are only to facilitate descriptions of the present invention and to simplify the descriptions, rather than to indicate or imply that the referred device or element is limited to the specific direction or to be operated or configured in the specific direction. Therefore, the above-mentioned terminologies shall not be interpreted as confine to the present invention.

It should be understood that the term “a” or “an” should be understood as “at least one” or “one or more”. In other words, in one embodiment, the number of an element may be one, and in another embodiment, the number of the element may be plural. The term “a” or “an” should not be understood as a limitation on the number.

The present invention provides an acoustic ear-hook. The acoustic ear-hook can be retained at a cavum conchae when using the audio function, and can be inverted and hooked on an earlobe when not used for listening the audio, that is, can provide the function of the earphone and the function of the decoration.

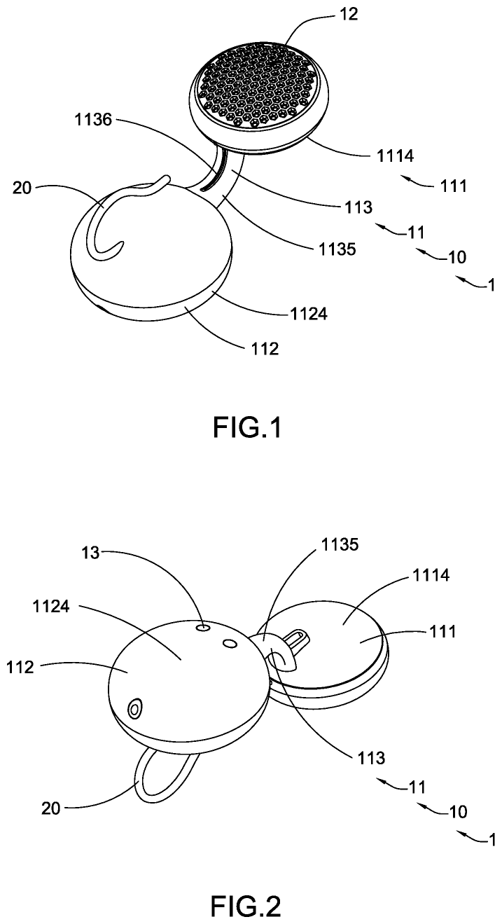

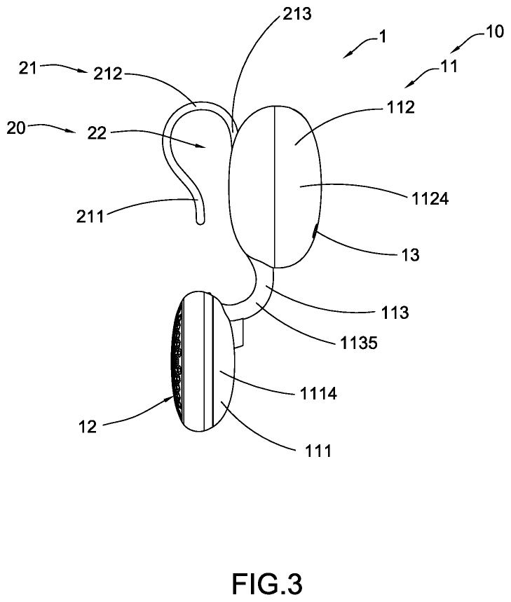

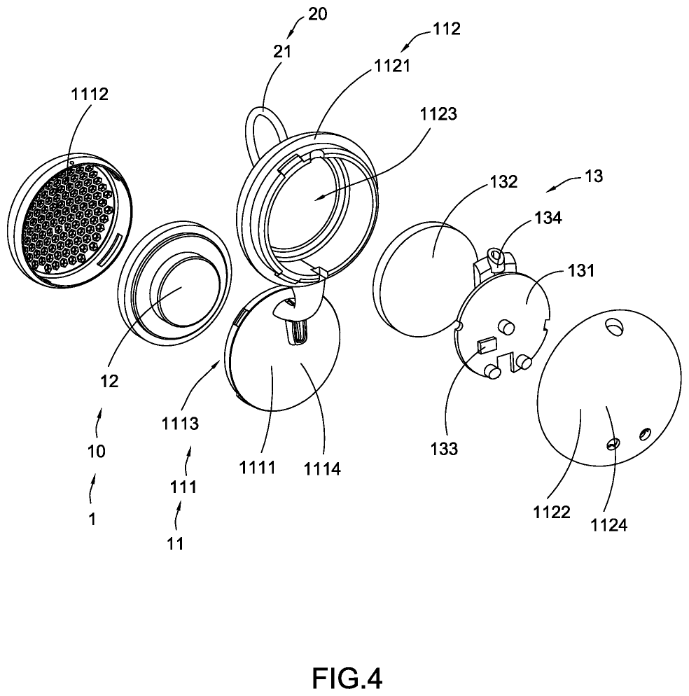

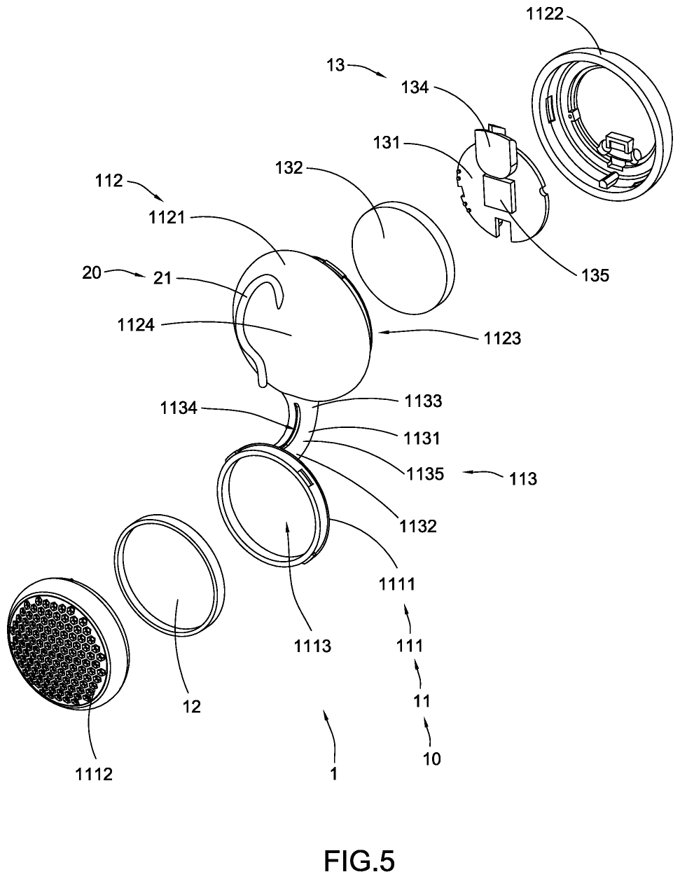

Referring to FIGS. 1 to 8 of the drawings, an acoustic ear-hook 1 according to a first preferred embodiment is illustrated, wherein the acoustic ear-hook 1 comprises an earphone assembly 10 and a hooking portion 20. The earphone assembly 10 is connected to the hooking portion 20 and can be control to provide an acoustic effect, and the hooking portion 20 is provided for hooking the acoustic ear-hook 1 on a user's ear 100.

The earphone assembly 10 is mainly used to controllably emit a sound, transmit sound to the user's ear 100, and enable the user to hear the sound emitted by its internal speaker. The earphone assembly 10 is adapted to be designed to communicate with an external device to obtain audio data and then emit the sound based on the audio data.

The hooking portion 20 is mainly used to hook on the user's ear 100, so that the acoustic ear-hook 1 can be entirely hooked on the user's ear 100, forming the decorative effect similar to that of an earring or ear-hook. Preferably, the hooking portion 20 is adapted to be hooked on an earlobe 101 of the user.

The earphone assembly 10 comprises a decorative shell 11, an acoustic part 12 and a control part 13. The acoustic part 12 is electrically connected to the control part 11, so as to generate the acoustic effect under control of the control part 11, and the acoustic part 12 and the control part 13 are respectively installed inside the decorative shell 11.

In addition, the hooking portion 20 is connected to the decorative shell 11.

The decorative shell 11 is mainly used to be installed with the acoustic part 12 and the control part 13 therein. In addition, the decorative shell 11 can also be provided with a surface for decorative design, such as diamond inlayering, patterning, engraving or detachable assembly of ornaments and other crafts, so that the acoustic ear-hook of the invention has the decorative function. Alternatively, the surface of the decorative shell 11 can also be designed into a variety of different shapes and appearances as required to provide a variety of decorative styles.

The acoustic part 12 is mainly used to controllably emit the sound by receiving an electrical signal of the control part 13 and converting the electrical signal into a sound wave to emit the sound. Through the voice part 12, the acoustic ear-hook 1 can have the acoustic function.

The acoustic part 12 is adapted to comprises a loudspeaker, such as a moving-coil loudspeaker, a moving-iron loudspeaker, a planar diaphragm loudspeaker or an electrostatic diaphragm loudspeaker, etc.

The control part 13 is mainly used to transmit the electrical signal to the acoustic part 12 to make the acoustic part 12 to generate sound, and also provide the electric energy required for the acoustic part 12. In addition, the control part 13 is also adapted to be communicated with the external device and obtain audio signals from the external device. The mode of communication between the control part 13 and the external device is adapted to be wired or wireless. The external device can be a mobile phone, a media player, or a computer. Moreover, the earphone assembly 10 can realize different functions by adding different working modules to the control part 13.

Preferably, the acoustic part 12 and the control part 13 are separately installed inside the decorative shell 11.

The decorative shell 11 comprises a first decorative body 111, a second decorative body 112 and a connecting element 113, wherein the first decorative body 111 and the second decorative body 112 are respectively connected to two ends of the connecting element 113.

On the other hand, the acoustic part 12 is positioned inside the first decorative body 111 in a manner which is capable of generating outward emitting sounds, and the control part 13 is positioned inside the second decorative body 112.

The first decorative body 111 is mainly used for a built-in installation of the acoustic part 12, so that the receiving structures of the acoustic part 12 the and control part 13 can be separated. The dispersed installation method of the acoustic part 12 can allow the acoustic part 12 to have a larger size for providing preferable sound quality.

Because the acoustic part 12 will be placed in the cavum conchae 102 of the user's ear 100, the acoustic part 12 needs to be matched with the cavum conchae 102, so the acoustic part 12 cannot be made large, if the control part 13 is also integrated, it will inevitably reduce the size of the acoustic part 12, and then affect the sound quality.

Therefore, the way in which the acoustic part 12 and the control part 13 are separated and installed in different decorative bodies is quite beneficial for improving the sound quality.

In addition, the outside surface of the first decorative body 111 can also be used by decorative processes or operations to make the appearance of the first decorative body 111 having the aesthetic value.

The first decorative body 111 can be placed at the cavum conchae 102 of the ear

100 in a semi-in-ear manner and abutted against the antitragus for sending sound to the ear of the user. The first decorative body 111 can also play a sound near the ear in a non-in-ear manner.

Preferably, the first decorative body 111 is implemented to substantially have a shape of a disk for better matching the acoustic part 12.

The second decorative body 112 is mainly used for the built-in installation of the control part 13, so that the control part 13 is independent of the acoustic part 12. This enables the control part 13 to have a larger size, in order that the control part 13 can be configured with the components having a larger number or size, such as batteries or various functional modules, also in order that the control part 13 can have the better expansibility. For example, the working life of the acoustic ear-hook 1 can be prolonged by increasing the size of the battery. For another example, adding more functional modules can enhance the application scenario of the acoustic ear-hook 1 and make it more functional.

In addition, the outside surface of the second decorative body 112 can also be made to have the aesthetic value through the decorative processes or operations.

Further, the internal space can be further enlarged by expanding the volume of the second decorative body 112 to accommodate the larger control part 13 to add or accommodate more components.

The connecting element 113 is mainly used for stably connecting the first decorative body 111 and the second decorative body 112, so that the connecting element 113, the first decorative body 111 and the second decorative body 112 form an integral structure.

Further, the outside surface of the connecting element 113 is also adapted to be made to have the aesthetic value through the decorative processes or operations.

The first decorative body 111 and the second decorative body 112 enable the acoustic part 12 and the control part 13 to be installed separately and independently so that each of the acoustic part 12 and the control part 13 can enhance their own effects, moreover, the connecting element 113 connects the first decorative body 111 and the second decorative body 112 to form a one-piece structure, and the earphone assembly 10 still forms an integrated structure, so that it has the overall stability.

Through the artistically machining for the outside surface of the first decorative body 111, the second decorative body 112 and the connecting element 113, the whole earphone assembly 10 can form the aesthetic appearance. On the surface, the acoustic ear-hook 1 is an earpiece, which has the function of an earphone at the same time, so that it gets rid of the monotony of the conventional earphones, and it also has the better expansibility than the conventional earphones.

The first decorative body 111 comprises a first shell 1111 and a first lid 1112, wherein the first decorative body 111 also has a first accommodating chamber 1113 and comprises a first decorative surface 1114. The first lid 1112 is assembled with the first shell 1111 in a manner which is capable of transmitting the sound. The first accommodating chamber 1113 is formed inside the first lid 1112 and the first shell 1111, and the first decorative surface 1114 is formed on the outside surface of the first decorative body 111.

In addition, the acoustic part 12 is positioned in the first accommodating chamber 1113 in the manner which is capable of outwardly emitting sounds, and the sound emitting side of the acoustic part 12 is positioned towards the first lid 1112.

The first shell 1111 and the first lid 1112 are combined to form the internal first accommodating chamber 1113, and the first accommodating chamber 1113 is provided for the separate installation of the acoustic part 12. The combined connection mode of the first shell 1111 and the first lid 1112 is also convenient to install the acoustic part 12 during the production process.

The first lid 1112 is mainly used for conducting the sound emitted by the acoustic part 12 outwardly.

The first decorative surface 1114 is mainly used to provide the surface of decorative design which can be designed on the first decorative surface 1114 by means of patterning, diamond inlaying or carving, etc., so that the first decorative surface 1114 has the decorative aesthetic effect.

Further, the outside surface of the first lid 1112 can also be decorated to enhance the decorative effect of the first decorative body 111.

The second decorative body 112 comprises a second shell 1121 and a second lid 1122. The second decorative body 112 also has a second accommodating chamber 1123 and comprises a second decorative surface 1124. The second lid 1122 is assembled to the second shell 1121. The second accommodating chamber 1123 is formed inside the second shell 1121 and the second lid 1122, and the second decorative surface 1114 is formed on the outside surface of the second decorative body 112.

In addition, the control part 13 is positioned in the second accommodating chamber 1123.

The second shell 1121 and the second lid 1122 are combined to form the internal second accommodating chamber 1123 for separately installing the control part 13. The combination connection mode of the second shell 1121 and the second lid 1122 is also convenient to install the control part 13 during the production process.

Further, the sizes of the second accommodating chamber 1123 can be increased by changing the sizes of the second shell 1121 and the second lid 1122, so that more or larger components can be installed inside the second decorative body 112. For example, the battery size can be increased to increase the battery life of the acoustic ear-hook 1. For another example, more functional modules are adapted to be added to provide a variety of different functions, so that it has better functional expansibility.

The second decorative surface 1124 is mainly used to provide a surface with the decorative design, wherein the surface of the second decorative surface 1124 can be designed with patterns, inlaying diamonds or carvings, etc., so that the second decorative surface 1124 has the decorative aesthetic effect.

The connecting element 113 comprises a connecting body 1131, a first end portion 1132 and a second end portion 1133, wherein the connecting element 113 further has a wire groove 1134 and comprises a third decorative surface 1135, wherein the first end portion 1132 and the second end portion 1133 are respectively formed at both ends of the connecting body 1131. The wire groove 1134 is arranged in the connecting body 1131, the wire groove 1134 is communicatively arranged between the first end portion 1132 and the second end portion 1133, and the third decorative surface 1135 is formed on the outside surface of the connecting element 113.

In addition, the first end portion 1132 is connected to the first shell 1111 of the first decorative body 111, and the second end part 1132 is connected to the second shell 1121 of the second decorative body 112.

The connecting body 1131 is mainly used to connect the first decorative body 111 and the second decorative body 112, so that the first decorative body 111 and the second decorative body 112 are connected to form an entire structure.

The first end portion 1132 is mainly used to connect the connecting body 1131 to the first shell 1111. The second end portion 1133 is mainly used to connect the connecting body 1131 to the second shell 1121. Through the connection of the first end portion 1132, the second end portion 1133 and the connecting body 1131, the first decorative body 111 and the second decorative body 112 can form a stable entire structure.

The wire groove 1134 is mainly used to arrange the wires 1136 connecting the acoustic part 12 and the control part 13. The wires 1136 are arranged in the wire groove 1134, for better protecting the wires and avoiding damage from the external force.

The third decorative surface 1135 is mainly used to provide a surface with the decorative design, wherein the third decorative surface 1135 can be designed with patterns, inlaying diamonds or carvings, etc., so that the third decorative surface 1135 has the decorative aesthetic effect.

Referring to the FIG. 8 of the drawings, by combining the first decorative surface 1114 of the first decorative body 111, the second decorative surface 1124 of the second decorative body 112 and the third decorative surface 1135 of the connecting element 113, a large part of the surface area of the acoustic ear-hook 1 can have a decorative design, and the acoustic ear-hook 1 is almost an ornament in appearance, so that it is capable of playing the role of the ornament and can also provide the function of the earphone.

Preferably, the material of the decorative shell 11 is copper, as copper is convenient for inlaying diamonds. The main material of the decorative shell 11 can also be stainless steel, silver, gold and other metals, can also be a variety of alloys, can also be non-metallic materials, such as ABP plastic, PP plastic, PC plastic, rubber, silicone and other materials. The material of the decorative shell 11 is adapted to be elastic or rigid.

The control part 13 comprises a control circuit 131, a power module 132, a communication module 133 and a charging module 134, wherein the control circuit 131 is electrically connected to the power module 132, the communication module 133 is electrically connected to the control circuit 131, and the charging module 134 is rechargeable electrically connected to the control circuit 131.

In addition, the acoustic part 12 is electrically connected to the control circuit 131, so as to generate sound under control of the control circuit 131.

The control circuit 131 is mainly used for overall control of the acoustic part 12, the communication module 133, the charging module 134 and other components. The control part 13 can receive the audio data from the communication module 133 and convert the audio data into the audio signals then transmit to the acoustic part 12 for generating sound under control. The control part 131 can control the charging or discharging of the charging module 134, and avoid the overcharging or over-discharging of the power module 132 for protecting the power module 132.

The power module 132 is mainly used to provide the electrical power for the control circuit 131, and then provide the electrical energy required for work to other components indirectly.

Preferably, the power module 132 can be repeatedly charged and discharged. The communication module 133 is mainly used for the communication connection with the external devices, such as mobile phones, media players, computers and other electronic devices. The communication module 133 receives the data sent by the external devices and transfers the data to the control circuit 131, so that the data is processed by the control circuit 131.

Preferably, the communication module 133 is wirelessly connected to the external device, such as by Bluetooth, WIFI and other wireless communication connection manners.

The charging module 134 is mainly used to charge the power module 132 through the external electrical power. The power module 132 can be used multiple times by charging.

The hooking portion 20 comprises a hooking body 21 and defines a hooking space 22, wherein the hooking body 21 is connected to the second shell 1121 of the second decorative body 112, the hooking space 22 is formed between the hooking body 21 and the second shell 1121 of the second decorative body 112.

The hooking body 21 is mainly used to be hooked on the earlobe 101 of the user's ear 100, and the hooking body 21 can pass through the ear hole at the earlobe 101 to connect the hooking portion 20 with the earlobe 101, so that the acoustic ear-hook 1 is hanged in the ear 100, furthermore, the acoustic ear-hook 1 can be entirely hooked to the user's ear 100.

The hooking space 22 is mainly used to provide a space to accommodate the user's ear 100, so that when the hooking body 21 is hooked on the ear 100, the hooking body 21 can also have an movement space.

Through the hooking body 21, the acoustic ear-hook 1 can be hooked on the user's ear 100, and through the hooking space 22, the acoustic ear-hook 1 can perform a certain form adjustment and activity at the user's ear 100.

The hooking body 21 comprises an entry end 211, an adjustment part 212 and a connecting end 213, wherein the connecting end 213 is connected to the second shell 1121, one end of the adjustment part 212 is connected to the connecting end 213, the other end of the adjustment part 212 is connected to the entry end 211.

The entry end 211 is mainly used to pass through the ear hole of the user's ear 100, in particular the ear hole at the earlobe 101, for hooking the hooking body 21 on the ear 100

The adjustment part 212 is mainly used to be hooked on the ear 100 and allows a certain amount of movement so that the hooking body 21 can be adjusted on the ear 100.

The connecting end 213 is mainly used to connect the hooking body 21 to the second shell 1121, so that the hooking body 21 is connected to the second shell 1121.

When using the acoustic ear-hook 1, the user hooks the acoustic ear-hook 1 on the ear 100 by passing the hooking body 21 of the hooking portion 20 through the ear hole of the ear 100. At this time, the acoustic ear-hook 1 can form the decorative effect of the earpiece, similar to the effect of the earring or ear hook.

When the user needs to listen to audio such as music and news, the control part 13 of the earphone assembly 10 can be connected with the external device, and the data can be sent to the communication module 133 of the control part 13 from the external device.

The communication module 133 transmits the received data to the control circuit 131, and the control circuit 131 controls the acoustic part 12 to generate sound after processing the data.

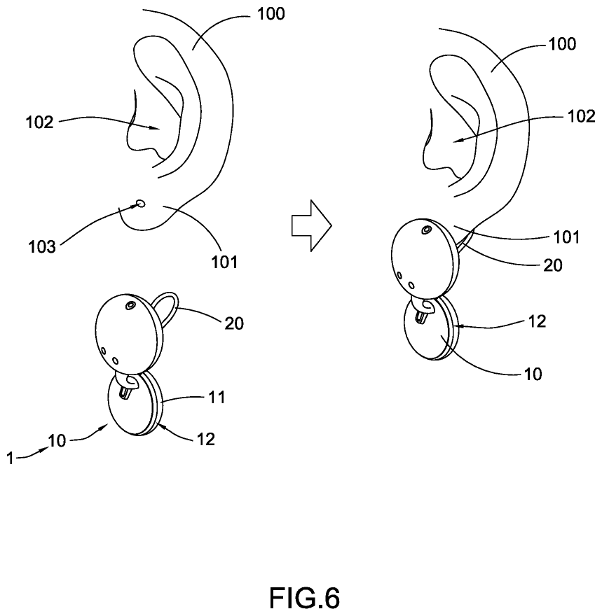

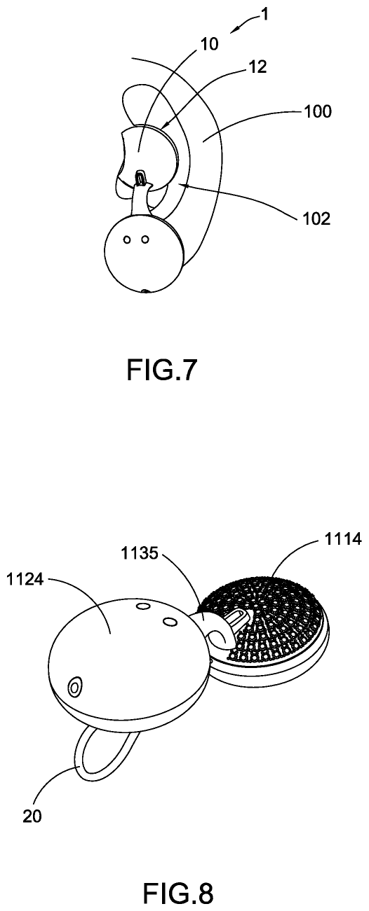

Referring to the FIGS. 6 to 7 of the drawings, the acoustic ear-hook 1 according to the first preferred embodiment of the present invention comprises at least two using modes.

One using mode is to hang it on the ear as the ornament, and play the sound around the ear through the acoustic part 12, so that the acoustic ear-hook 1 forms the playing mode in the non-in-ear manner.

This non-in-ear playing mode will be more hidden, and not easy to cause others to notice, so that it is adapted to be applied to some public occasions, and the user is able to listen to audio messages without attracting the attention of others.

Preferably, when hanging the acoustic ear-hook 1 as the ornament, the acoustic ear-hook 1 does not work to generate sound. In other words, when the user does not want to use the acoustic ear-hook 1 as the earphone for listening to the acoustic sound, as shown in FIG. 6 of the drawings, the hooking body 21 of the hooking part 20 is hooked on the earlobe 101 of the ear 100 by passing the hooking body 21 of the hooking portion 20 through the ear hole of the earlobe 101 of the ear 100, so that the second decorative body 112 is attached to the earlobe 101 and the first decorative body 111 is hung below the earlobe 101 of the user to function as a decoration element.

The other using mode is that adjust the acoustic ear-hook 1 by rotating or folding, and place the acoustic part 12 at the cavum conchae 102 for sound play, the earlobe 101 is received in the hooking space 22 and is clamped between the hook body 21 and the second shell 1121 of the second decorative body 112, as shown in FIG. 7 of the drawings, so that the acoustic ear-hook 1 forms the semi-in-ear playing mode.

This semi-in-ear playing mode provides better sound quality and enables the acoustic ear-hook 1 to be stably fixed at the cavum conchae 102. In this using mode, the acoustic ear-hook 1 can be in a certain movement without falling, and stably provide the acoustic effect for the user.

The user can conveniently switch between the two modes mentioned above because the adjustment part 212 of the hooking body 21 and the hooking space 22 provide sufficient space for the acoustic ear-hook 1 to adjust.

The two different playing modes mentioned above can provide different audio experience, and different playing modes can also form different decorative effects.

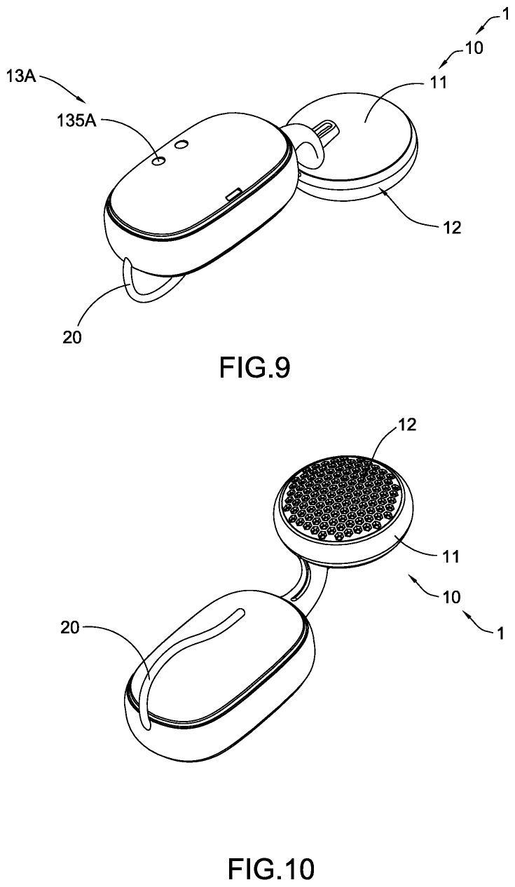

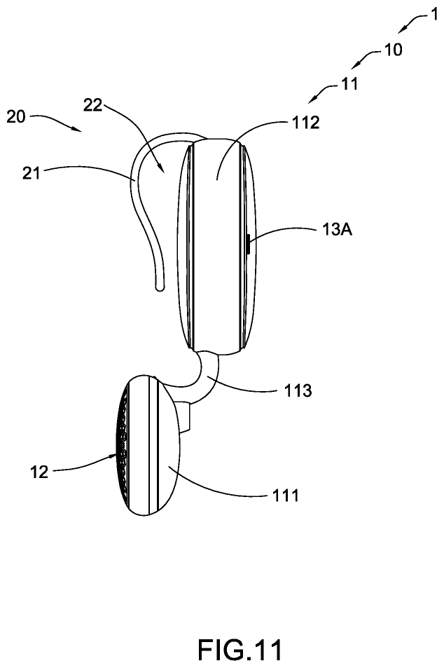

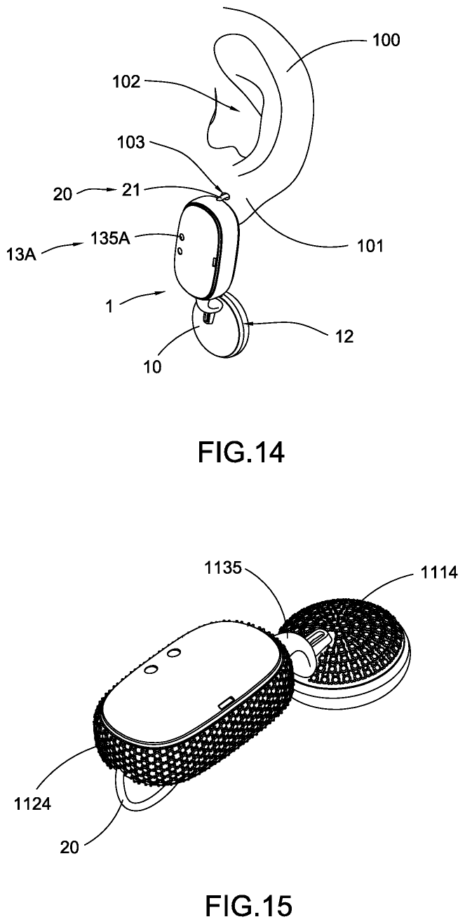

Referring to FIGS. 9 to 15 of the drawings, an acoustic ear-hook 1A according to a second preferred embodiment of the present invention is illustrated, wherein the acoustic ear-hook 1A has a similar structure with the above first preferred embodiment. The acoustic ear-hook 1A comprises the earphone assembly 10 and the hooking portion 20, wherein the earphone assembly 10 is connected to the hooking portion 20 in a controllable vocal manner. The hooking portion 20 is provided for hooking the acoustic ear-hook 1 to the user's ear 100.

The difference is that in the second preferred embodiment, the decorative shell 11 of the earphone assembly 10 comprises the first decorative body 111, a second decorative body 112A and the connecting element 113, wherein the first decorative body 111 and the second decorative body 112A are respectively connected at both ends of the connecting element 113.

In addition, the acoustic part 12 is positioned inside the first decorative body 111 in the manner which is capable of outwardly emitting sound, and the control part 13 is positioned inside the second decorative body 112 which has a rectangular shape.

Wherein, the volume of the second decorative body 112A is increased compared with that of the first preferred embodiment, which enables the second decorative body 112A to have a second accommodating chamber 1123A with a larger volume than that of the first preferred embodiment and can be used to install a control part 13A with a larger volume.

The larger control part 13A can add more function modules to the control circuit 131, so that the acoustic ear-hook 1 can have more functions.

The larger power module 132 can also be installed to provide a longer battery life.

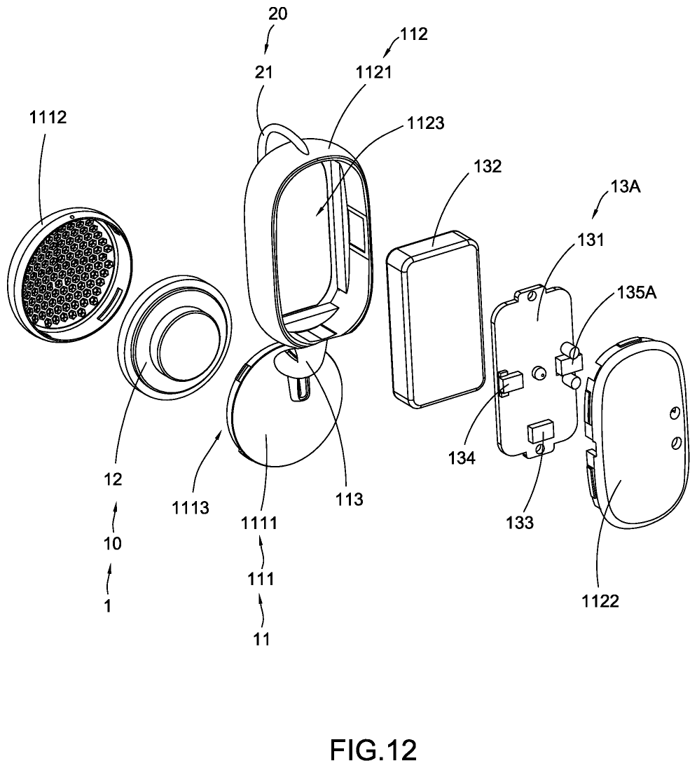

Referring to the FIG. 12 of the drawing, based on the first preferred embodiment, the control part 13A further comprises a microphone unit 135A, which is electrically connected to the control circuit 131 in a manner which is capable for receiving voice of the user.

The microphone module 135A is mainly used to receive the surrounding voice of the user and transmit the received voice to the control circuit 131.

Preferably, the microphone module 135A is a miniature microphone.

The acoustic ear-hook 1 has the function of making phone calls, such that when the user makes the phone call with others, the user's voice content can be received by the acoustic ear-hook 1, and the data related to the voice content is sent to the external device through the control circuit 131 and the communication module 133. Finally, the data of the voice content is sent to the device of the communication object by the external device.

In the opposite direction, the external device receives the voice content of the communication object, sends the data related to the voice content to the control part 13A, and finally is played by the acoustic part 12.

In addition, after the microphone unit 135A has received external voice of the user, the microphone unit 135A can amplify the volume through the control circuit 131, and finally play the amplified sound signals through the acoustic part 12, which enables the acoustic ear-hook 1A to have the function of a hearing-aid.

More specifcially, in the case that the control circuit 131 is integrated with a hearing-aid module, the hearing aid module comprises the microphone unit 135A an amplifier, a receiver and other structures, wherein the microphone unit 135A is used to collect sound and convert it into electric waves, the amplifier is used to increase the intensity of the electrical waves, and the receiver is used to convert the electrical waves back into the sound waves. The acoustic ear-hook 1A can also switch between a phone mode and a hearing-aid mode. When a call comes in, the acoustic ear-hook 1A is switched into the phone mode at this time and the hearing-aid mode can be used in normal times, so that it is convenient for the user with poor hearing to wear, and can avoid the trouble caused by frequent replacement of earphone and hearing-aid.

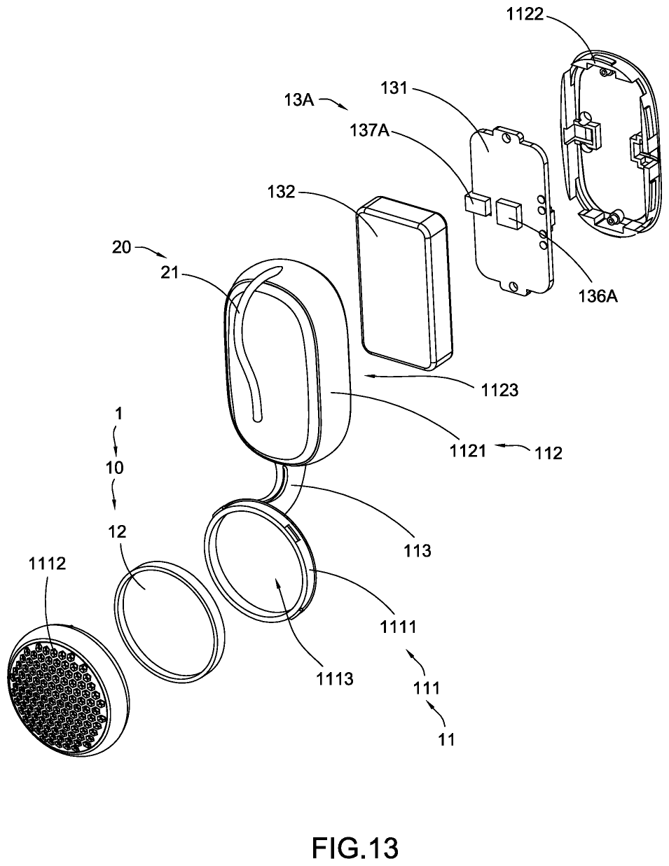

Further, referring to the FIG. 13, the control part 13 comprises an artificial intelligence module 136A electrically connected to the control circuit 131.

The user can dictate the voice command to the artificial intelligence module 136A, the voice information is collected by the microphone unit 135A, and then is identified through the artificial intelligence module 136A, and finally operation according to the identified content is implemented.

The artificial intelligence module 136A can be interacting with the user, such as feedbacking the search network information to the user, performing the online shopping, playing audio, making phone calls, sending emails, controlling home equipments or office equipments, etc.

Furthermore, the control part 13 further comprises a heart rate detection module 137A electrically connected to the control circuit 131 for detecting a heart rate.

The heart rate detection module 137A can detect the user's heart rate and transmit the heart rate data to the control circuit 13 which can correspondingly perform the operation based on the heart rate data.

Preferably, the heart rate detection module 137A is a photoelectric sensor for measuring the heart rate. By measuring the optical changes in the blood in response to the pulse, the heart rate is detected and estimated.

Studies have shown that it is possible to predict depression with 90% accuracy by analyzing the person's heart rate over a 24-hour period. The researchers found that the depressed patients generally had the higher heart rates and the lower heart rate variability, by using these heart rate data, the artificial intelligence applications can be used to correctly identify the depressed patients and the non-depressed participants.

The heart rate detection module 137A monitors the user's heart rate and other datum, and then analyzes the datum through the control circuit 131 or the artificial intelligence module 136A. If a higher possibility is identified, the control circuit 131 can play the preset music or imitate predetermined sound, so as to adjust and improve the depressed mood to keep the user in a happy state.

When detecting a higher possibility of the user suffering from depression, the acoustic ear-hook 1A can also notify a guardian through the external device software, such as a mobile phone application, so that the guardian can make further examination in time and detect the condition early.

When producing and assembling the acoustic ear-hook of the present invention, the first shell 1111, the connecting element 113 and the second shell 1121 can be formed as a whole piece, and then the acoustic part 12 is installed in the first accommodating chamber 1113, and the control part 13 is installed in the second accommodating chamber 1123, respectively, meanwhile, the wires 1136 are set in the wire groove 1134 of the connecting part 113 to electrically connect the acoustic part 12 and the control part 13, finally, the first lid 1112 is assembled with the first shell 1111, and the second lid 1122 is assembled with the second shell 1121.

In addition, the decorative operations can be carried out on the outside surface of the decorative shell 11 before, during or after assembly, such as diamond inlaying, engraving and other processes.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and are subject to change without departure from such principles. Therefore, this invention includes all modifications encompassed within the spirit and scope of the following claims.

What is claimed is:

1. An acoustic ear-hook, comprising:
an earphone assembly; and
a hooking portion, wherein said earphone assembly is connected to said hooking portion for providing an acoustic effect, said hooking portion is provided for hooking said acoustic ear-hook to a user's ear.

2. The acoustic ear-hook, as recited in claim 1, wherein said earphone assembly comprises a decorative shell, an acoustic part and a control part, wherein said acoustic part is electrically connected to said control part to provide the acoustic effect under control of said control part, wherein said acoustic part and said control part are respectively installed inside said decorative shell, and said hooking portion is connected to said decorative shell.

3. The acoustic ear-hook, as recited in claim 2, wherein said decorative shell comprises a first decorative body, a second decorative body and a connecting element, wherein said first decorative body and said second decorative body are respectively connected to two ends of said connecting element, and said acoustic part is positioned inside said first decorative body in a manner which is capable of outwardly emitting sound, wherein said control part is positioned inside said second decorative body.

4. The acoustic ear-hook, as recited in claim 3, wherein said first decorative body comprises a first shell and a first lid, wherein said first decorative body also has a first accommodating chamber and comprises a first decorative surface, wherein said first lid is assembled with said first shell in a manner which is capable of transmitting the sound, wherein said first accommodating chamber is formed inside said first lid and said first shell, and said first decorative surface is formed on an outside surface of said first decorative body, said acoustic part is positioned in said first accommodating chamber, and an acoustic side of said acoustic part is positioned towards said first lid.

5. The acoustic ear-hook, as recited in claim 4, wherein said second decorative body comprises a second shell and a second lid, wherein said second decorative body has a second accommodating chamber and comprises a second decorative surface, wherein said second lid is assembled to said second shell, wherein said second accommodating chamber is formed inside said second shell and said second lid, and said second decorative surface is formed on an outside surface of said second decorative body, wherein said control part is positioned in said second accommodating chamber.

6. The acoustic ear-hook, as recited in claim 5, wherein said connecting element comprises a connecting body, a first end portion and a second end portion, wherein said connecting element further has a wire groove and comprises a third decorative surface, wherein said first end portion and said second end portion are respectively formed at two ends of said connecting body, wherein said wire groove is indented in said connecting body, and said third decorative surface is formed on an outside surface of said connecting element, wherein said first end portion is connected to said first shell of said first decorative body, and said second end portion is connected to said second shell of said second decorative body.

7. The acoustic ear-hook, as recited in claim 3, wherein said hooking portion comprises a hooking body which is connected to said second decorative body, and a hooking space is formed between said hooking body and said second decorative body.

8. The acoustic ear-hook, as recited in claim 7, wherein said hooking body comprises an entry end, an adjustment part and a connecting end, wherein said connecting end is connected to said second decorative body, one end of said adjustment part is connected to said connecting end, the other end of said adjustment part is connected to said entry end.

9. The acoustic ear-hook, as recited in claim 3, wherein said first decorative body is adapted to be placed at a cavum conchae of the ear and abutted against an antitragus.

10. The acoustic ear-hook, as recited in claim 2, wherein at least part of said decorative shell is provided with decorative elements by diamond inlaying, patterning, engraving or detachable assembly of an ornament.

11. The acoustic ear-hook, as recited in claim 1, wherein said control part comprises a control circuit, a power module, a communication module and a charging module, wherein said control circuit is electrically connected to said power module, said communication module is electrically connected to said control circuit, and said charging module is rechargeable electrically connected to said control circuit, wherein said acoustic part is electrically connected to said control circuit to provide the acoustic effect under control of said control circuit.

12. The acoustic ear-hook, as recited in claim 11, wherein said control part further comprises a microphone unit which is electrically connected to said control circuit in a manner which is capable for receiving voice of the user.

13. The acoustic ear-hook, as recited in claim 12, wherein said control part further comprises an artificial intelligence module electrically connected to said control circuit.

14. The acoustic ear-hook, as recited in claim 11, wherein said control part further comprises a heart rate detection module electrically connected to said control circuit.

15. The acoustic ear-hook, as recited in claim 1, wherein said acoustic ear-hook integrates functions of hearing-aid and earphone.

16. The acoustic ear-hook, as recited in claim 1, wherein said earphone assembly comprises a decorative shell, an acoustic part and a control part, wherein said acoustic part is electrically connected to said control part, wherein said decorative shell comprises a first decorative body, a second decorative body and a connecting element which is extended between said first decorative body and said second decorative body, wherein said acoustic part is disposed ins said first decorative body which is capable of being placed at a cavum conchae of the ear for enabling said acoustic part to provide the acoustic effect to the user's ear and being hung under an earlobe of the ear when said hooking portion is hooked on the ear.

17. The acoustic ear-hook, as recited in claim 16, wherein when said first decorative body is placed at the cavum conchae, said hooking portion and said second decorative body define a hooking space for clamping the earlobe of the user.

18. The acoustic ear-hook, as recited in claim 16, wherein when said hooking portion is penetrating the earlobe of the user for retaining said second decorative body on the earlobe, said first decorative body is hung under said first decorative body.

19. The acoustic ear-hook, as recited in claim 16, wherein said control part is disposed in said second decorative body.

20. The acoustic ear-hook, as recited in claim 16, wherein at least one of said first decorative body, said connecting element and said second decorative body is provided with decorative elements by diamond inlaying, patterning, engraving or detachable assembly of an ornament.
